(19) United States
(12) Patent Application Publication
(10) Pub. No.: US 2020/0267949 A1
(43) Pub. Date: Aug. 27, 2020

(54) Control System for a Pet Enclosure Lamp With Sound Simulation of an Ecological Environment

(71) Applicant: Zoo Med Laboratories, Inc., San Luis Obispo, CA (US)

(72) Inventor: Gary Bagnall, San Luis Obispo, CA (US)

(73) Assignee: Zoo Med Laboratories, Inc.

(21) Appl. No.: 16/287,067

(22) Filed: Feb. 27, 2019

(51) Int. Cl. A01K 63/06; H05B 37/02; H05B 33/08; A01K 63/00; A01K 1/035; H04R 1/02; H04R 3/00; A61N 5/06

(52) U.S. Cl. CPC A01K 63/06; H05B 37/0272; H05B 37/0254; H05B 33/0845; A01K 63/006; A01K 1/035; A61N 2005/0652; H04R 3/00; A61N 5/0613; A61N 2005/0661; A61N 2005/0654; A61N 2005/0626; A61N 2005/0663; H04R 1/028

(57) ABSTRACT

A control system for a pet enclosure lamp with sound simulation of an ecological environment includes a control module, an audio module, and dimming control circuitry. A pet enclosure lamp is also provided. The control system can be used with any type of pet.

### BACKGROUND OF THE INVENTION

### 1. Field of the Invention

The present invention relates to the field of pet enclosure light control. In particular, the invention relates to a pet enclosure light control system integrating sound simulation of the ecological environment being simulated in the pet enclosure.

### 2. Description of the Related Art

Pet enclosure lamps, a special kind of luminaire, provide light for the growth and development of plants and animals in the pet enclosure by replacing the natural sunlight with synthesized sunlight according to the needs of the plants and animals. The pet enclosure lamp can emit super white light, making the water body transparent and clear, and the plant color vivid. At the same time, the pet enclosure lamp emitting super white light can promote the photosynthesis of plants and promote plant growth and development. Super white light has a good effect on green plants and is suitable for plant growth with water depths below 50 cm to 60 cm.

Some pet enclosure lamps are like sunlight, suitable for a variety of plants and fish growth. These pet enclosure lamps have a strong light penetration, which makes the fish color more vivid. These pet enclosure lamps are mainly suitable for providing the best illumination for pet enclosure landscapes.

Commonly, pet enclosure lamps are composed of four types of LED lights, namely red light, blue light, medium color warm light, and low color temperature light. After the fish is irradiated by the light, the color is bright, the fish body is fresh, and the water body is transparent and clear. Red light is especially suitable for the lighting of red goldfish, blood parrot and other fish species.

Blue light, after the fish is illuminated by this light, is azure in color, as clear and realistic as sea water. Blue light is especially suitable for corals, shrimps and other organisms. Blue light can promote the absorption of coral calcium and synthesize vitamin D3 to make it grow healthily and brightly.

A pet enclosure lamp mixed with medium and low color temperature light can make the lamp emit soft, reddish light, and promote the growth and development of plants. Under the light of a pet enclosure lamp mixed with medium and low color temperature light, the plants of various colors are realistic, and the branches are thick and strong, which is more suitable for a mixed plant environment. A pet enclosure lamp mixed with medium and low color temperature light is especially suitable for plants mixed with red and green. It has a good effect on red plants. It is more suitable for tanks with water depths below approximately 60 cm, and the effect is better.

At present, pet enclosure lamps on the market have the following defects: the function is relatively simple; the lighting environment is not convenient to control; and it is impossible to simulate ecological sounds and bring a more convenient environment for plants and animals.

### SUMMARY OF THE INVENTION

It is, therefore, an object of the present invention to provide a control system for a lamp in a pet enclosure with sound simulation of an ecological environment. The control system includes a control module, an audio module, and dimming control circuitry. A lamp is also provided.

It is also an object of the present invention to provide a control system for a lamp in a pet enclosure with sound simulation of an ecological environment further including a power regulator.

It is another object of the present invention to provide a control system for a lamp in a pet enclosure with sound simulation of an ecological environment wherein the control module includes a single-chip control microprocessor.

It is a further object of the present invention to provide a control system for a lamp in a pet enclosure with sound simulation of an ecological environment wherein the audio module includes a sound processing microprocessor and associated audio circuitry.

It is also an object of the present invention to provide a control system for a lamp in a pet enclosure with sound simulation of an ecological environment further including simulated sounds maintained on a solid-state memory.

It is another object of the present invention to provide a control system for a lamp in a pet enclosure with sound simulation of an ecological environment further including a wireless remote controller.

It is a further object of the present invention to provide a control system for a lamp in a pet enclosure with sound simulation of an ecological environment further including a wired remote controller.

It is also an object of the present invention to provide a control system for a lamp in a pet enclosure with sound simulation of an ecological environment wherein the lamp includes only white LED lights and blue LED lights.

It is another object of the present invention to provide a control system for a lamp in a pet enclosure with sound simulation of an ecological environment wherein the lamp includes red, green & blue LED lights, white LED lights, blue LED lights and UVA.

It is a further object of the present invention to provide a control system for a lamp in a pet enclosure with sound simulation of an ecological environment wherein the audio module includes a sound processing microprocessor, an audio output interface, an external speaker access detection circuit, and a USB interface.

Other objects and advantages of the present invention will become apparent from the following detailed description when viewed in conjunction with the accompanying drawings, which set forth certain embodiments of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are top plain views of alternate embodiments of a control system for a pet enclosure lamp with sound simulation of an ecological environment.

FIG. 2 is a schematic of the control system in accordance with an alternative embodiment of the present invention.

FIG. 3 is a circuit diagram of a single-chip control microprocessor of the control module for control system for a pet enclosure lamp with sound simulation of an ecological environment.

FIG. 4 is a circuit diagram of a synchronous depressurization power regulator of the control system for a pet enclosure lamp with sound simulation of an ecological environment.

FIG. 5 is a circuit diagram of audio circuitry of the sound processing microprocessor of the control system for a pet enclosure lamp with sound simulation of an ecological environment.

FIG. 6 is a circuit diagram of an external speaker access detection circuitry of the control system for a pet enclosure lamp with sound simulation of an ecological environment.

FIG. 7 is a circuit diagram of an LED dimming control circuit diagram of the control system for a pet enclosure lamp with sound simulation of an ecological environment.

FIG. 8 is a circuit diagram of a debugging interface circuitry of the control system for a pet enclosure lamp with sound simulation of an ecological environment.

FIG. 9 is a circuit diagram of an external control interface circuitry of the control system for a pet enclosure lamp with sound simulation of an ecological environment.

FIG. 10 is a control schematic diagram of the control system of a pet enclosure lamp with sound simulation of an ecological environment.

FIGS. 11A-11D show various light spectrum profiles in accordance with the embodiment disclosed with reference to FIG. 1A.

FIGS. 12A-12G show various light spectrum profiles in accordance with the embodiment disclosed with reference to FIG. 1B.

FIG. 13 is an alternate pet enclosure lamp that may be used in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

Referring to the various figures, the present invention provides a control system 10 for a pet enclosure lamp 12 (in particular, an LED pet enclosure lamp in accordance with disclosed embodiments) with sound simulation of an ecological environment. While the term “pet enclosure lamp” is used throughout the present disclosure, it is appreciated that the lamp and control system may be used in a variety of environments and in conjunction with a variety of fish, reptiles or animals. As will be explained in more detail, the control system 10 operates in conjunction with a 24V power supply 14, and the control system 10 generally includes a power regulator 16, a control module 18 including a single-chip control microprocessor U3, an audio module 19 including a sound processing microprocessor U4 and associated audio circuitry, LED dimming control circuitry 20. As will be appreciated based upon the following disclosure, the various components of the control system 10 operate in a coordinated manner under the control of the control module 18. Further, and as will be appreciated based upon the following disclosure, the audio module 19 includes a sound processing microprocessor U4 composed of a single-chip sound processing microprocessor U4 that processes the simulated sound of the ecological environment for output. In accordance with a preferred embodiment, and as is well known to those skilled in the art, the simulated sound(s) are maintained on a solid-state memory 22 housed on the sound processing microprocessor U4. The built-in sound data includes the simulation of ecological environmental sound, such as, ocean waves, waves lapping upon reefs, waves lapping upon the beach, thunderstorms, rain dropping, etc. These sounds enable pet enclosure owners to enjoy a more realistic experience with their pets within the pet enclosure environment.

More specifically, the control system 10 of the present invention allows pet enclosure owners to combine lighting effects and audio effects to create a highly productive pet enclosure environment. As mentioned above, the control system 10 offers eight unique audio tracks, although it is fully appreciated more or less audio tracks may be integrated for use in conjunction with the present invention.

In conjunction with the audio tracks, the control system 10 allows for selection of various lighting modes, for example, the control system 10 can be set to operate in a 24 hour mode with customizable day and night light settings and special sunset and sunrise transitions. In addition, the control system 10 of the present invention may be used to enhance the tank's environment with mesmerizing effects such as waves, fading clouds, and lightning storms, all with their own synchronized sounds. In addition to the preprogrammed lighting and audio effects, the control system 10 allows users to custom program the control module 16 to allow a user to take advantage of the lights of the pet enclosure lamp 12 to customize and save preferred color settings for the pet enclosure 12.

Some of the preprogrammed modes of the present control system 10 are as follows:

Static Modes

SUN: All lights on. (Sound—Flowing river)

MOON: Blue light only. (Sound—Nighttime lake)

FISH: White, blue, and UV only. (Sound—Lake waves)

PLANT: All light on with additional red brightness. (Sound—Jungle stream)

Dynamic Effects

WAVES: Gentle waves crashing onto the shore.

MOON and CLOUDS: Clouds passing in front of a full moon.

CLOUDS: Overcast skies with cloud motion.

STORM: Thunderstorm and lightning.

The present control system is provided with either a wireless remote controller 100 or a wired remote controller 100′ allowing a user to fully control the tank's sound and appearance in a convenient manner.

As briefly mentioned above, the control system 10 works in conjunction with a pet enclosure lamp 12. The pet enclosure lamp 12 is powered and controlled by the control system 10 of the present invention and is therefore connected thereto using conventional wiring 80.

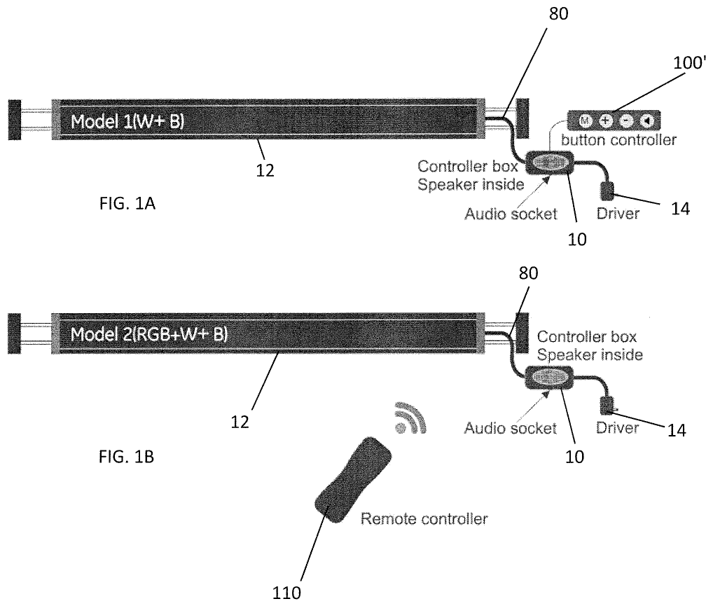

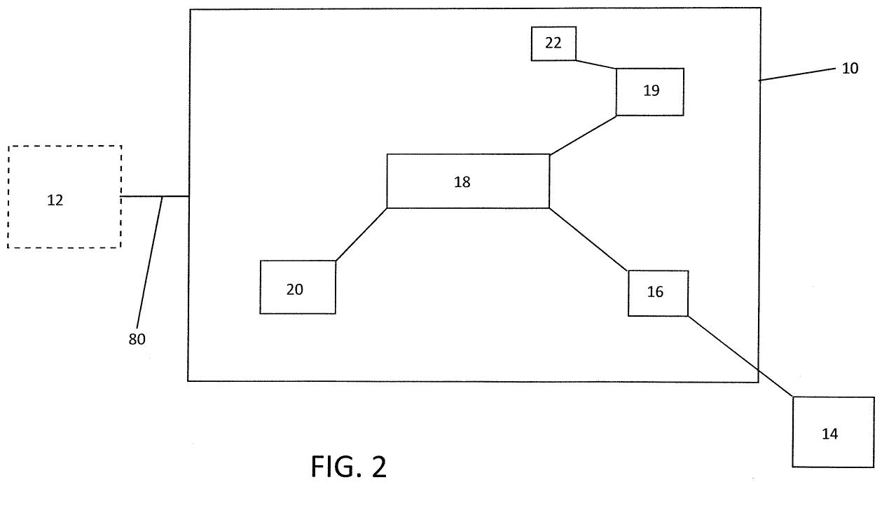

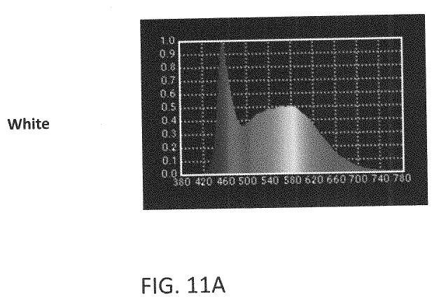

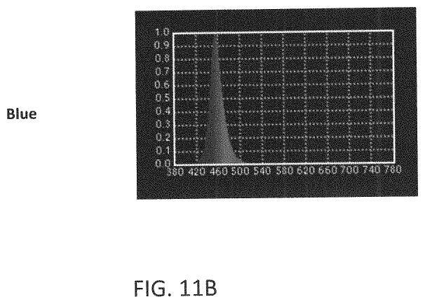

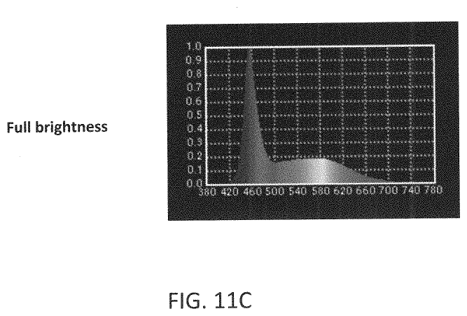

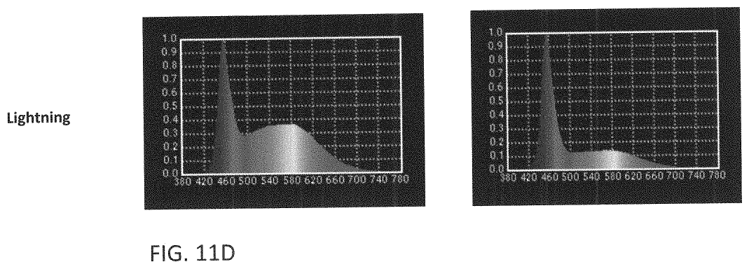

As will be explained below in more detail, and by way of example, it is contemplated the control system 10 may be used in conjunction with a simplified pet enclosure 12 including only white LED lights and blue LED lights (see FIG. 1A). In accordance with this embodiment, the LED pet enclosure 12 is capable of producing light with the light spectrum profiles shown in FIGS. 11A-11D, wherein FIG. 11A is a static white light profile, FIG. 11B is a static blue light profile, FIG. 11C is a static full brightness light profile, and FIG. 11D is a dynamic lightening light profile.

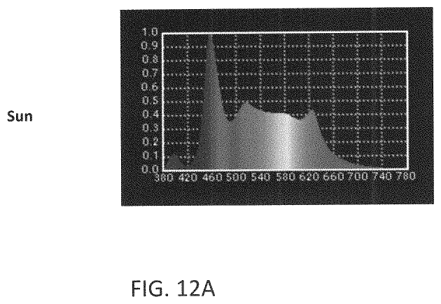

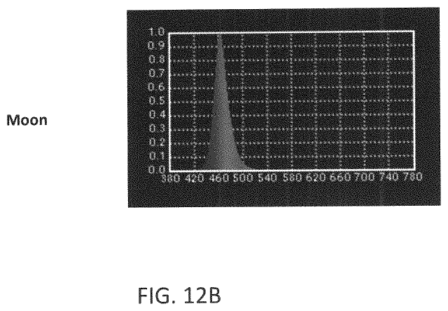

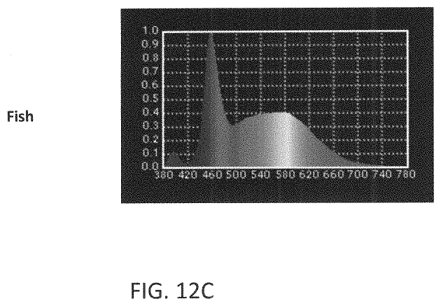

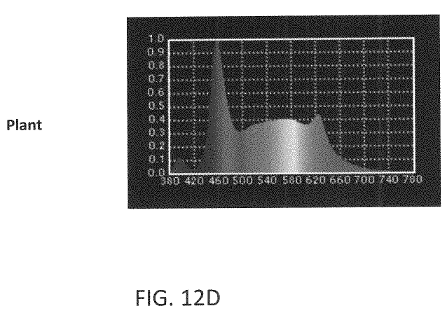

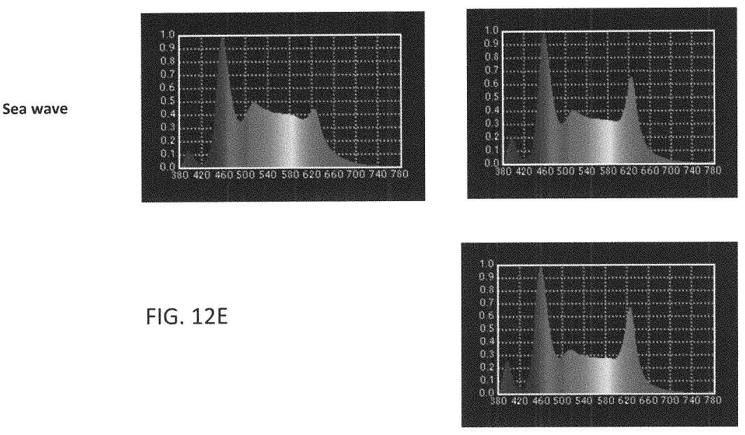

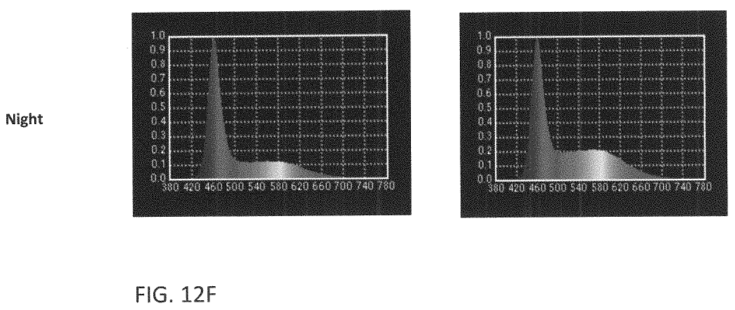

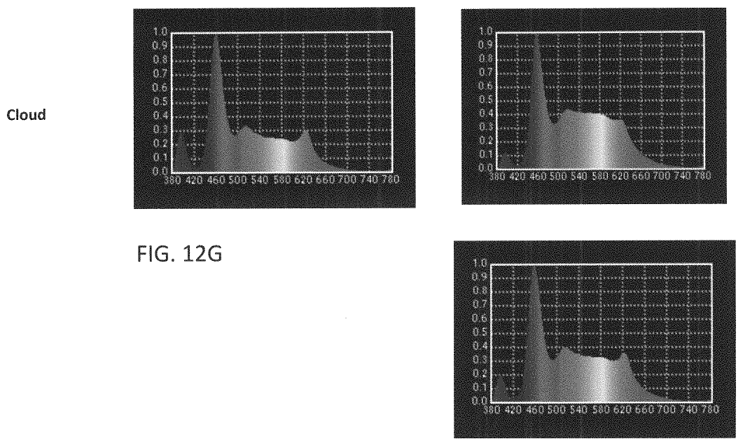

In accordance with another embodiment, the control system 10 may be used in conjunction with a more sophisticated pet enclosure lamp 12 including red, green & blue LED lights, white LED lights, and blue LED lights and UVA (see FIG. 1B). In accordance with this embodiment, the LED pet enclosure lamp 12 is capable of producing light with the spectrum profiles shown in FIG. 12A-12G, wherein FIG. 12A is a static sun light profile, wherein FIG. 12B is a static moon light profile, wherein FIG. 12C is a static fish light profile, wherein FIG. 12D is a static plant light profile, wherein FIG. 12E is a dynamic sea wave light profile, wherein FIG. 12F is a dynamic night light profile, wherein FIG. 12G is a dynamic cloud light profile.

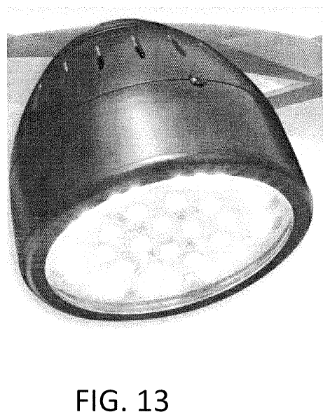

While exemplary pet enclosure lamps are disclosed above, it is appreciated that the pet enclosure lamp may take a variety of shapes and utilize various light producing mechanism. For example, and in addition to LED pet enclosure lamps, the light producing mechanism could be incandescent, fluorescent, etc. In addition, the shape of the light could be varied; for example, see the circular light shown in FIG. 13.

Regardless of the pet enclosure lamp, the control system 10 includes a full array of functionalities and the control system 10 is described below in conjunction with the latter embodiment. In addition, and regardless of the pet enclosure lamp, the present system provides the tank with the benefits of UVA light, which helps promote natural behavior and breeding in fish, in particular, it can increase breeding behavior, feeding response, and coloration in some shallow water fish. The latter embodiment provides lighting synched to distinctive audio tracks for 8 unique lighting modes as described above to mimic natural environments (jungle stream, lake waves, nighttime lake, flowing river, thunderstorm, overcast skies with rolling clouds, clouds passing in front of a full moon, and gentle waves). The more budget friendly embodiment features 5 lighting modes that incorporate unique audio tracks for an immersive pet enclosure experience for the user (lake waves, jungle stream, nighttime lake, light rain and waves, and thunderstorm audio effects).

As will be appreciated based upon the following disclosure, the 24V power supply 14 provides power to the control system 10 and the pet enclosure lamp 12 via the power regulator 16. The power regulator 16 converts the 24V power supply 24 into a 5V power supply, and then supplies power to the various elements of the control system 10 and the pet enclosure lamp 12. After the audio module 19 powers up and connects with the control module 18, the simulated ecological environmental sound stored in a solid-state memory 22 (which, in accordance with a preferred embodiment, is an independent chip) is accessed, processed and output by the audio module 19. As explained above, the built-in sound data includes the sound of the waves rolling, the sound of the waves hitting the reef, the sound of the waves hitting the beach, the sound of lightning, the raindrops when raining, and the sound of the simulated ecological environment. The LED dimming control circuitry 20 is connected to the control module 18, in particular, the single chip control microprocessor U3 of the control module 18 and is connected to the pet enclosure lamp 12.

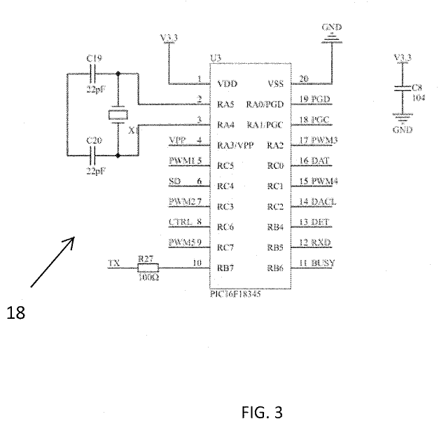

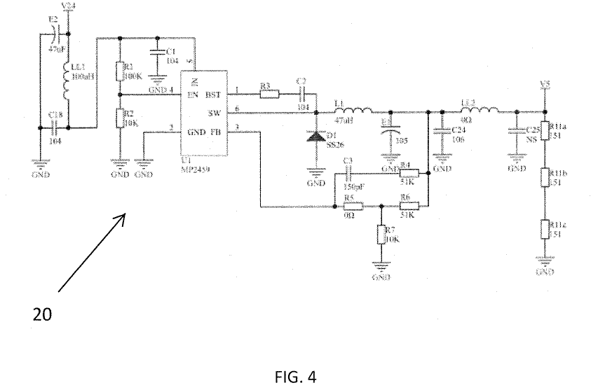

Referring to FIG. 3, the control module 18 includes a single-chip control microprocessor U3 is Model No. PIC16F18345. The 1 pin of the single-chip control microprocessor U3 is connected with the voltage output terminal V3.3 of the circuitry of the power regulator 16. The 2 pin and the 3 pin of the single-chip control microprocessor U3 are connected to a clock circuit 26. The 5, 7, 9, 17, and 15 of the single-chip control microprocessor U3 are respectively connected to the LED dimming control circuitry 20. The 14-pin of the single-chip control microprocessor U3 is connected with the audio output interface U6 of the audio module 19. The 18-pin and 19-pin of the single-chip control microprocessor U3 are connected with a communication interface J2 for on-chip debugging of the single-chip control microprocessor U3. The 20-pin connects the single-chip control microprocessor U3 to ground.

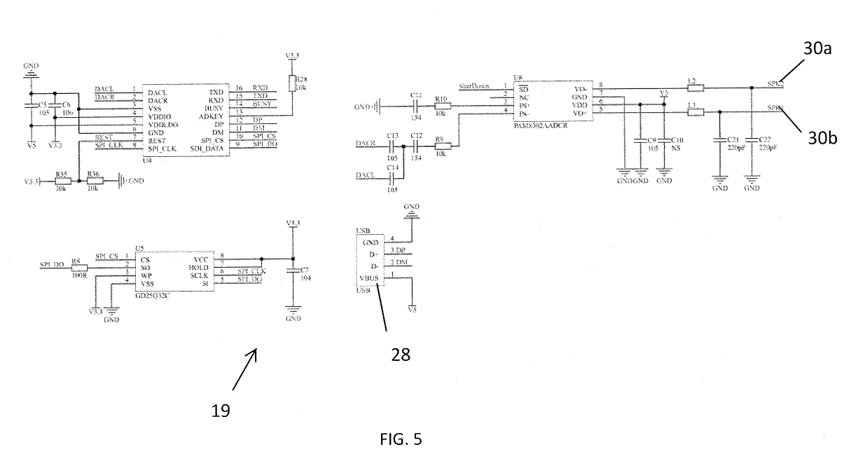

Referring to FIG. 4, the power regulator 16 includes a capacitors C1, C2, C3, C18, C24, C25, polar capacitors E1, E2, resistors R1, R2, R3, R4, R5, R7, R11a, R11b, R11c, inductors L1, LL1, LL2, diode D1, and a linear regulator U1. The linear regulator U1 is model of MP2459. The 1 pin and 6 pin of the linear regulator U1 are connected to the voltage output terminal V5. The capacitors C24 and C25, as well as polar capacitor E1, are connected in parallel, one end is grounded and one end is connected to the voltage output terminal V5. Capacitor C2 is connected to the 1 pin with resistor R3 therebetween. Diode D1 is connected with one end connected to the 6 pin and the other end connected to ground. Resistors R4, R5, and R6, as well as capacitor C3 are connected between the 3 pin and the voltage output terminal V5. Further, resistors R11a, R11b, and R11c are connected in series with one end connected to the voltage output terminal V5 and the other end connected to ground. Finally, the 4 pin and 5 pin are connected to the 24V power supply 14 with capacitors C1 and C18, polar capacitor E2, resistors R1 and R2, and inductor LL1 therebetween.

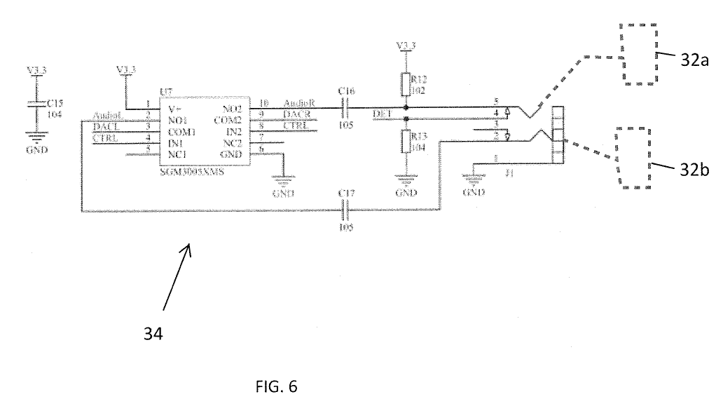

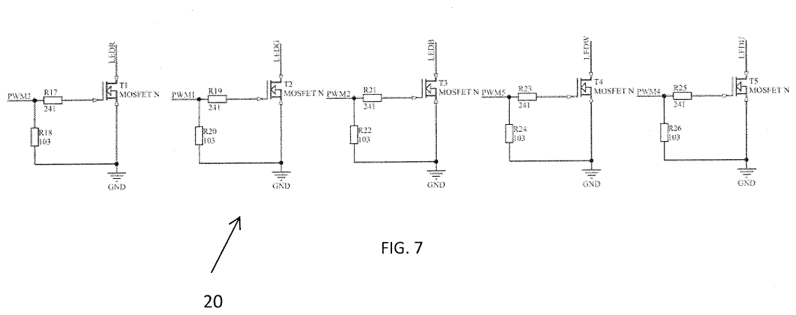

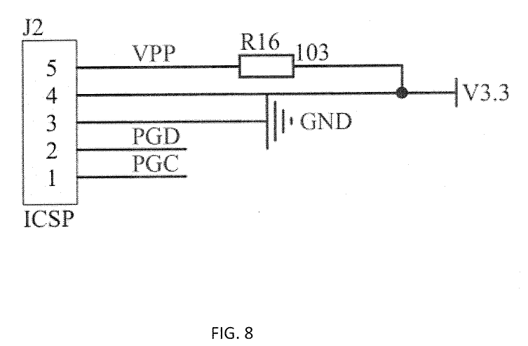

Referring to FIGS. 5 and 6, the audio module 19 of the present control system 10 is composed of a sound processing microprocessor U4, audio output interface U6, clock U5, an external speaker access detection circuit 34, and a USB interface 28. The 16 pin of the sound processing microprocessor U4 is connected to the 10 pin of the single-chip control microprocessor U3 through the resistor R27. The 1 pin and the 2 pin of the sound processing microprocessor U4 are respectively connected to the audio output interface U6. The 3 pin and the 6 pin of the sound processing microprocessor U4 are grounded.

In addition to the audio output interface U6 being connected to the sound processing microprocessor U4 via the 1 pin and the 2 pin of the sound processing microprocessor U4, the 5 pin and the 6 pin of the audio output interface U6 are connected to the first onboard speaker 30a of the control system 10 and the 8 pin is connected to the second onboard speaker 30b of the control system 10.

Further still, a USB interface 28 is connected to the sound processing microprocessor U4 via the 11 pin and the 12 pin of the sound processing microprocessor U4. The USB interface 28 allows for connection of external speakers 32a, 32b to the control system 10. The USB interface 28 may also be used for burning audio files.

With this in mind, the audio module 19 further includes the external speaker access detection circuit 34 as shown in FIG. 6. The external speaker access detection circuit 34 allows for automated attachment of the external speakers 32a, 32b to the audio module 19 in a manner facilitating convenient switching between the built in onboard speaker(s) 30a, 30b and external speaker(s) 32a, 32b.

Referring to FIG. 7, LED dimming control circuitry 20 includes resistors R17, R18, R19, R20, R21, R22, R23, R24, R25, R26 and field effect transistors T1, T2, T3, T4, T5. The source of the FET T1 is connected to the LEDR. The drain of the FET T1 is grounded, and the resistor R18 is provided between the drain of the FET T1 and the 17 pin of the control microprocessor 18. A resistor R17 is provided between the gate of the FET T1 and the 17 pin of the control microprocessor 18.

The source of the FET T2 is connected to the LEDG. The drain of the FET T2 is grounded, and the resistor R20 is provided between the drain of the FET T2 and the 5 pin of the control microprocessor 18. A resistor R19 is provided between the gate of the FET T1 and the 5 pin of the control microprocessor 18

The source of the FET T3 is connected to the LEDB. The drain of the FET T3 is grounded, and the resistor R22 is provided between the drain of the FET T3 and the 7 pin of the control microprocessor 18. A resistor R21 is provided between the gate of the FET T1 and the 7 pin of the control microprocessor 18.

The source of the FET T4 is connected to the LEDW. The drain of the FET T4 is grounded, and the resistor R24 is provided between the drain of the FET T4 and the 9 pin of the control microprocessor 18. A resistor R23 is provided between the gate of the FET T1 and the 9 pin of the control microprocessor 18.

The source of the FET T5 is connected to the LEDU. The drain of the FET T5 is grounded, and the resistor R26 is provided between the drain of the FET T5 and the 15 pin of the control microprocessor 18. A resistor R25 is provided between the gate of the FET T1 and the 15 pin of the control microprocessor 18.

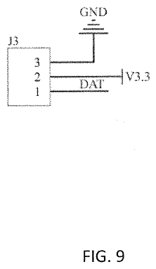

Referring to FIG. 9, the control system 10 of the pet enclosure lamp 12 with simulated ecological environment sound also includes an external control interface J3. The external control interface J3 pin 1 is connected with the 16 pin of the single chip control microprocessor U3. The external control interface J3 pin 2 is grounded. The external control interface J3 is 3 pin and the output of the power regulator circuit.

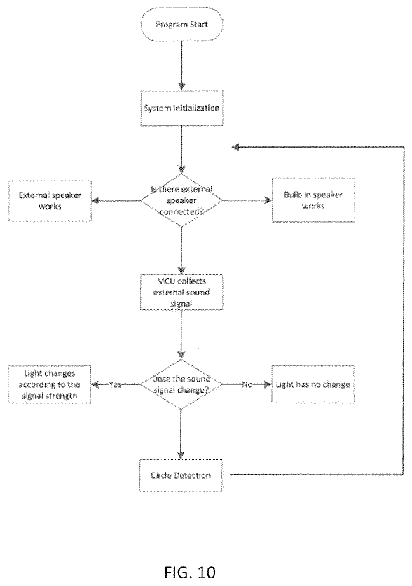

In order to better describe the solution of the present application, the control principle of the present solution will be described. As shown in FIG. 10, the program starts and the control system 10 is initialized. The control system 10 first determines if an external speaker(s) 32a, 32b is connected. If an external speaker(s) 32a, 32b is connected, the external speaker(s) 32a, 32b will be powered via the control system 10 and audio signals will be sent thereto. If no external speaker(s) is detected, the built-in speaker(s) 30a, 30b will be powered via the control system 10 and audio signals will be sent thereto.

The control system 10 then determines if there is a change in the signal strength. If a change in the sound signal strength is identified, the pet enclosure lamp 12 is adjusted based upon the external sound signal strength. If no change in the sound signal strength is identified, the pet enclosure lamp 12 remained the same.

The control system 10 then continually cycles to determine whether the sound signal strength and/or the status of an external speaker have changed. If a change is detected, the control system 10 acts as described above.

Since the control module 18 is connected to the LED dimming control circuitry 22 and the audio module 19 at the same time, the control program pre-fabricated in the control module 18 allows the control module 18 to control the LED illumination offer by the pet enclosure lamp 12 and can control the audio module 19 to play the corresponding sound effect separately or simultaneously.

With the foregoing in mind, the present control system provides an immersive lighting system for home pet enclosures that combines dynamic lighting and audio effects via a built-in speaker to simulate weather events and specific environments or locations. The ever changing color blend of the LEDs combined with immersive audio tracks help create a unique experience for the user.

While the preferred embodiments have been shown and described, it will be understood that there is no intent to limit the invention by such disclosure, but rather, is intended to cover all modifications and alternate constructions falling within the spirit and scope of the invention.

1. A control system for lamp in a pet enclosure with sound simulation of an ecological environment, comprising:
a control system including a control module, an audio module, and dimming control circuitry; and
a lamp.

2. The control system for a lamp in a pet enclosure with sound simulation of an ecological environment according to claim 1, further including a power regulator.

3. The control system for a lamp in a pet enclosure with sound simulation of an ecological environment according to claim 1, wherein the control module includes a single-chip control microprocessor.

4. The control system for a lamp in a pet enclosure with sound simulation of an ecological environment according to claim 1, wherein the audio module includes a sound processing microprocessor and associated audio circuitry.

5. The control system for a lamp in a pet enclosure with sound simulation of an ecological environment according to claim 1, further including simulated sounds maintained on a solid-state memory.

6. The control system for a lamp in a pet enclosure with sound simulation of an ecological environment according to claim 1, further including a wireless remote controller.

7. The control system for a lamp in a pet enclosure with sound simulation of an ecological environment according to claim 1, further including a wired remote controller.

8. The control system for a lamp in a pet enclosure with sound simulation of an ecological environment according to claim 1, wherein the lamp includes only white LED lights and blue LED lights.

9. The control system for a lamp in a pet enclosure with sound simulation of an ecological environment according to claim 1, wherein the lamp includes red, green & blue LED lights, white LED lights, blue LED lights and UVA lights.

10. The control system for a lamp in a pet enclosure with sound simulation of an ecological environment according to claim 1, wherein the audio module includes a sound processing microprocessor, an audio output interface, an external speaker access detection circuit, and a USB interface.
